(19) United States
(12) Patent Application Publication
(10) Pub. No.: US 2024/0382730 A1
(43) Pub. Date: Nov. 21, 2024

(54) FLEXIBLE MICRONEEDLE ARRAY PATCH FOR CHRONIC WOUND OXYGENATION AND BIOFILM ERADICATION

(71) Applicant: Purdue Research Foundation, West Lafayette, IN (US)

(72) Inventor: Rahim Rahimi, West Lafayette, IN (US)

(73) Assignee: Purdue Research Foundation, West Lafayette, IN (US)

(21) Appl. No.: 18/691,452

(22) PCT Filed: Sep. 14, 2022
(86) PCT No.: PCT/US2022/043522

§ 371 Date: Mar. 12, 2024

Related U.S. Application Data
(60) Provisional application No. 63/243,876, filed on Sep. 14, 2021.

(51) Int. Cl. A61M 37/00; A61K 9/00; A61K 33/08; A61K 47/32; A61K 47/34

(52) U.S. Cl. CPC A61M 37/0015; A61K 9/0021; A61K 33/08; A61K 47/32; A61K 47/34; A61M 2037/0023; A61M 2037/0053

(57) ABSTRACT

A medical patch may include a flexible substrate and a plurality of water-soluble microneedles extending from a surface of the substrate. The water-soluble microneedles may include polyvinylpyrrolidone (PVP) and calcium peroxide (CPO). Application of the medical patch to a wound causes the PVP to dissolve and the CPO to interact with the water and form hydrogen peroxide and oxygen.

### TECHNICAL FIELD

This disclosure relates to medical devices and, in particular, to wound dressing.

### BACKGROUND

Chronic wounds annually cost the worldwide economy $25 billion and is predicted to rapidly increase due to prevalence of obesity, ageing population, and increasing health care costs. These wounds are generally defined as slow-healing wounds due to complex bacterial colonies that cause infections and develop into biofilms, among other complications. Bacterial biofilms consist of social networks and structurally organized aggregates of bacteria and necrotic tissue that strongly attach to the tissue surface. As bacteria attach to the tissue surface, they produce a polysaccharide matrix that enhances their attachment and forms a physical barrier on the wound surface, causing an increased resistance to antibiotics, sustained immune response, and local hypoxia. Localized hypoxia results from an increased consumption of oxygen from both biofilms and local tissue as the wound attempts to heal. However, unlike healthy wounds, the elevated oxygen consumption is sustained due to increased bacterial proliferation within the wound, leading to a halt in the natural wound healing process.

One of the main challenges associated with biofilms is their increased antimicrobial resistance primarily resulting from the reduced penetration of antibiotics within the biofilm. Because traditional therapeutics are thus largely ineffective to treat chronic wounds, debridement is commonly used to remove biofilms and necrotic tissues to allow oxygen levels to return to normal and reduce risk of infection. Additionally, the removal of the biofilm layers allows for administration of antibiotics to eradicate any remaining bacteria. Although this process has many positive effects on the wound status, there are still several drawbacks. Debridement must be performed often, as biofilms and necrotic tissue continually reform until chronic wounds heal. Also, debridement is painful, non-selective, and often requires additional surgical procedures to revascularize tissue. Additional antibiotics to help combat infection, or alternative therapies to improve tissue oxygenation, such as hyperbaric oxygen therapy are also often required after debridement procedures.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates an example of a medical patch.

FIG. 2A-G illustrates an example of the laser processing and polymer micromolding process for fabrication of a medical patch.

FIG. 3 illustrates a T-peel bonding test between PET and PVP for plasma-treated and nonplasma-treated samples with various concentrations of photoinitiator.

FIG. 4 illustrates TGA curves of the commercial PVP polymer, UV polymerized PVP, and the UV-polymerized PVP-CPO composite.

FIG. 5 illustrates a Micro-CT image taken of one MN loaded with CPO.

FIG. 6 illustrates Average force for a single MN to pierce through pig skin tissue and fracture against the rigid PMMA surface.

FIG. 7 of the oxygen generation test of MNs loaded with CPO.

FIG. 8A-B illustrates a bactericidal study on planktonic bacteria with (A) Log 10 CFU mL⁻¹ of S. aureus and aeruginosa monitored when exposed to PET (control), MNs without CPO, and MNs loaded with CPO.

FIG. 9A-B illustrates bactericidal studies on 1 week old biofilm.

FIG. 10 illustrates an ex vivo assay performed against P. aeruginosa and S. aureus on a pig skin-infected wound model.

FIG. 11 Illustrates bar graphs of cell survival percentage over time under different conditions

### DETAILED DESCRIPTION

Hyperbaric oxygen therapy increases the level of oxygen within the wound to help promote the natural wound-healing process of the body and has been shown to promote healing in severe chronic wounds. However, many of such treatments require often bulky equipment and expose large areas of the body to an unnecessarily elevated oxygen level that can damage healthy tissue. Hence, a localized, widely accessible method for selectively increasing the levels of oxygen within the wound combined with an antibacterial agent would be advantageous compared to conventional techniques. For this new method to be effective, it would need to be able to bypass the biofilm barrier to effectively administer the therapeutic agents and increase the level of oxygen within the wound.

Biodegradable microneedles (MNs) would be able to provide the vehicle for the therapeutic agents as the MN structure would pierce through the top layer (biofilm and cellular debris) and dissolve upon contact with biological fluid and thus release the therapeutics inside the wound. MN platforms have been commonly studied as options for vaccines, antibiotics, and drug delivery vehicles for their ability to bypass the stratum corneum without the pain associated with hypodermic needles. They also have higher drug efficacy than hypodermic needles due to direct injection into the bloodstream rather than muscle tissue. MNs created from nonbiodegradable materials such as metal or silicone have distinct disadvantages as compared to biodegradable polymer-based ones. Nonbiodegradable materials introduce risk of needles breaking off into the body, disease transmission through reuse, and create hazardous waste. Conversely, biodegradable polymeric materials degrade upon application, leaving behind no hazardous waste and disallowing reuse.

Although some studies have investigated the use of MNs for the treatment of wound biofilms, most of these studies have utilized antibiotics that either produce harsh side effects or only had a slight impact on the biofilms. Additionally, the manufacturing processes used do not permit the fabrication of flexible MN array patches, thus limiting their application to flat surfaces as their rigid substrate cannot properly conform to a curved surface on the human body. Furthermore, the therapeutics used by these studies do not address the inherent hypoxia of the chronic wounds, thus still requiring the use of additional treatments to fully address the complex nature of the wound.

Accordingly, there is disclosure and device and manufacturing methods for a flexible microneedle array patch. By way of an introductory example, a medical patch may include a substrate and a plurality of water-soluble microneedles. The microneedles may, together, form a flexible polymer composite microneedle array that can overcome the physicochemical barriers (i.e., bacterial biofilm) present in chronic nonhealing wounds and co-deliver oxygen and bactericidal agents. The polymeric microneedles are made by using a facile UV polymerization process of polyvinylpyrrolidone (PVP) and calcium peroxide (CPO) onto a flexible polyethylene terephthalate (PET) substrate for conformable attachment onto different locations of the human body surface.

The microneedles may elevate the oxygen levels from 8 to 12 ppm once dissolved over the course of 2 h while also providing strong bactericidal effects on both liquid and biofilm bacteria cultures of both Gram-positive (Staphylococcus aureus) and Gram-negative (Pseudomonas aeruginosa) bacterial strains commonly found in dermal wounds. Furthermore, results from an ex vivo assay on a porcine wound model indicated successful insertion of the microneedles into the tissue while also providing effective bactericidal properties against both Gram-positive and Gram-negative within the complex tissue matrix. Additionally, the microneedles demonstrate high levels of cytocompatibility with less than 10% of apoptosis throughout 6 days of continuous exposure to human dermal fibroblast cells. The demonstrated flexible microneedle array can provide a better approach for increasing the effectiveness of topical tissue oxygenation as well as the treatment of infected wounds with intrinsically antibiotic resistant biofilms.

The MNs are highly water soluble and upon exposure to water, CPO degrades into hydrogen peroxide and oxygen to kill bacteria present in the wound and oxygenate local tissue to promote wound healing, respectively. The MNs may also permit direct administration of therapeutics across a biofilm layer while simultaneously disrupting the architecture of the biofilm for higher antibiotic efficacy.

Additional benefits, efficiencies, and improvements over existing market solutions are made evident in the systems and methods described herein and in the Appendix attached hereto.

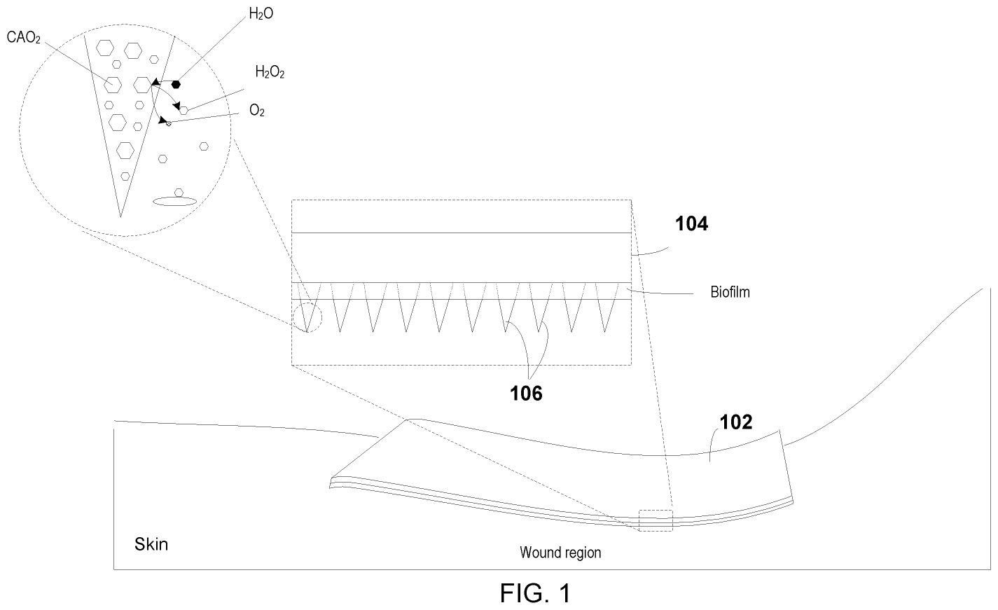

FIG. 1 illustrates an example of a medical patch 102 applied to a wound. The medical patch may include a flexible substrate 104. The substrate may include, for example, PET, or some other suitable material. The flexibility of the substrate 104 allows for conformability to the skin of a subject.

The medical patch 102 may include microneedles needles (MNs) 106 extending from a surface of the substrate 104. The microneedles may be water soluble and include an antibacterial substance. The antibacterial substance may be any which diminishes bacteria microbial infections. The microneedles may extend from the surface of the substrate 0.5 mm-2.5 mm.

In some examples, the MNs may be include polyvinylpyrrolidone (PVP) loaded with calcium peroxide (CAO₂). Since the MNs water soluble, upon exposure to water, the CAO₂ degrades into hydrogen peroxide and oxygen to kill bacteria present in the wound and oxygenate local tissue to promote wound healing, respectively. In some examples, the MNs may also allow for direct administration of therapeutics across a biofilm layer while simultaneously disrupting the architecture of the biofilm for higher antibiotic efficacy.

As illustrated in FIG. 1, the medical patch 102 may be applied to a wound. The microneedles may puncture a biofilm that forms on the surface of the wound. As the MNs dissolve, the CaO₂ reacts with H₂O at the wont region, releasing H₂O₂ and O₂

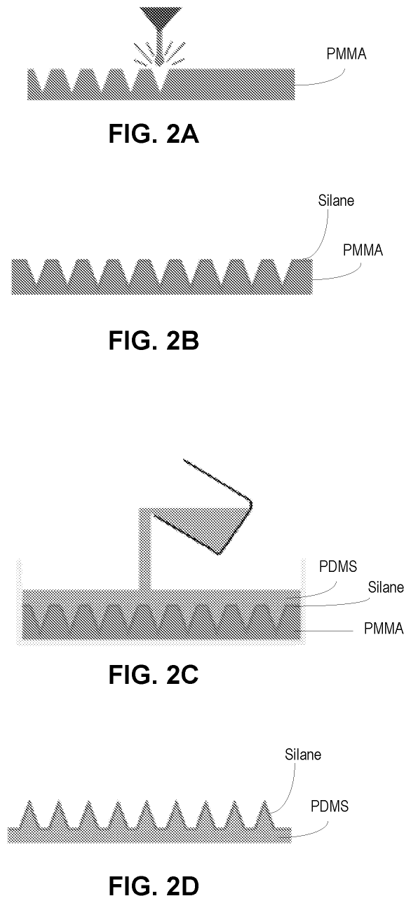

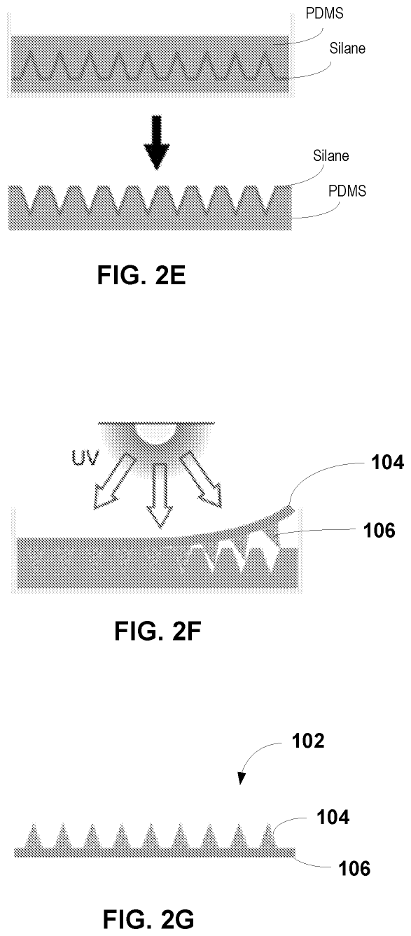

FIGS. 2A-G illustrate an example of the laser processing and polymer micromolding process for fabrication of the medical patch. FIG. 2A illustrates forming a MN master mold. The MN Master mold may be laser ablated out of a mold substrate 202 including, for example, a clear poly (methyl methacrylate) (PMMA) substrate. The ablation may performed in many ways, including with a CO₂ (10.6 μm) laser (PLS6MW, Universal Lasers, Inc., Scottsdale, AZ), with the focus offset by 3 mm and operated at a speed of 0.4 m s⁻¹ and a power of 54 W (FIG. 2a). In other examples, focus, speed, and power may be varied. The laser-ablated acrylic master mold may then be washed with distilled water and dried with nitrogen gas to remove dust and particles from the surface.

FIG. 2B illustrates application of silane to the acrylic master mold. The acrylic master mold may be silanized with, for example, 0.5 mL of (3-aminopropyl) triethoxysilane (Sigma-Aldrich, St. Louis, MO) under vacuum for 8 h.

FIG. 2C illustrates casting polydimethylsiloxane (PDMS) onto the silanized acrylic mold. In various experiments, PDMS (10:1 ratio, Sylgard 184, Dow Corning) was degassed to remove air trapped in MN holes and subsequently cured in the oven at 70° C. for 4 h.

FIG. 2D illustrates an example of the silanized MN array ater detachment from the acrylic mold. The flexible PDMS MN array may be gently detached from acrylic master mold and silanized using, for example, 0.5 mL (3-aminopropyl) triethoxysilane under vacuum for 8 h.

FIG. 2E illustrates forming a reusable mold for the medical patch. A second PDMS prepolymerized solution (e.g. 10:1 ratio) may be casted onto the silanized PDMS MNs, followed by degassing and curing in the oven at, for example, 70° C. for 4 h. The self-assembled silane layer creates a barrier between the PDMS MNs and PDMS prepolymerized solution, thus preventing the chemical bonding between the two PDMS layers during the PDMS-to-PDMS replication. After curing, the final PDMS mold may be carefully peeled off the PDMS MNs and silanized according to the previous process.

FIG. 2F illustrates forming the microneedles using the reusable mold. MNs loaded with CPO (Sigma-Aldrich, St. Louis, MO) may be fabricated using an optimized photopolymerization process between, for example, N-vinylpyrrolidone (NVP) (Sigma-Aldrich, St. Louis, MO), Irgacure 651 (Ciba Specialty Chemicals, Switzerland), and 10 wt % CPO. By way of example, after dissolving 0.4 wt % photoinitiator in NVP, 2 mL of the solution may be pipetted into the PDMS mold to cover the bottom of the mold and degassed to remove air from the MN holes. The photoinitiator may include IRGACURE 2959, or some other suitable photoinitiator. Next, 0.5 mL of a 10 wt % CPO solution in NVP may be sonicated for 10 min then added to the 3 cm by 3 cm female PDMS mold. After the CPO settled, excess NVP solution may be removed from the surface of the mold and a PET film that was plasma treated for 2 min may be carefully placed on top of the female mold. Following placement of the PET film, the mold may be irradiated with UV light (e.g. Sunray 600, Uvitron, MA, USA) for 30 min. The fully polymerized MNs loaded with CPO may be carefully peeled out of the mold and stored in a nitrogen box until further characterization FIG. 2G illustrates an example of the medical patch after it is removed from the reusable mold.

Optimization of the PVP Photopolymerization Process. The amount of photoinitiator used in polymerization directly impacts both the mechanical properties of the polymer produced and the duration of polymerization. Additionally, the photoinitiator concentration will influence the strength of interfacial bonding between the PET films and PVP MNs. However, because high concentrations of photoinitiator can be toxic to cells,₂₉ the minimum amount of photoinitiator should be used while still maintaining fast polymerization (<30 min) and good mechanical properties. Therefore, to determine the optimal concentration of the photoinitiator to use for polymerization and bonding to the PET substrate, we performed a T-peel test using an ADMET universal tensile machine at a jog rate of 0.5 mm min₋₁. To fabricate the samples, 10 μL of NVP solution with different photoinitiator concentrations (0, 0.1, 0.2, 0.4, 0.8, and 1.6 wt %) was dispensed at the end of a 1 cm by 6 cm strip of the PET film. A second strip of the PET film of the same dimensions was carefully laid upon the first strip with a spacer between the two strips to restrict the NVP solution to a 1 cm area between the two PET films. Next, the strips were irradiated with UV light at 100 W m₋₂ for 30 min. The process was repeated with PET films that were plasma treated using a plasma treater (PE-25, Plasma Etch, Inc., NV) under O₂ for 2 min at 200.3 mTorr. The samples were stored in a nitrogen box until use.

MN Characterization Due to the cytotoxicity and carcinogenic characteristics of many monomers, it is essential to verify full polymerization of PVP using the established photo polymerization process. Thermal degradation characterization was performed as a means to determine the possible remnants of the monomer within the UV-polymerized PVP samples. In this test, thermogravimetric analysis (TGA) was performed upon commercially purchased PVP (Sigma-Aldrich, St. Louis, MO) as the control and UV-polymerized PVP with and without CPO to compare the degradation behavior of all three materials. In this experiment, approximately 5 mg of each sample was analyzed using a TG 209 F3 Tarsus. All samples were heated to a maximum temperature of 750° C. at a rate of 20° C. per minute under a flow of nitrogen gas. MNs were imaged with optical microscopy and micro computed tomography (micro-CT). For optical microscopy, images were taken using an Olympus SZ2-ILST microscope (Olympus, Japan) at 20×203 magnification.

To analyze the distribution of CPO within the MN polymeric matrix, micro-CT imaging was conducted using a desktop micro-CT system (SkyScan 1272, Bruker, Billerica, MA). The X-ray beam energy was tuned to 35 kV/231 μA with no filter to optimize transmission. Two-dimensional (2D) projections were acquired with frame averaging set to 3 and rotation at steps of 0.2° over 180° to increase the signal-to-noise ratio. These radiographs were recorded with a camera resolution of 7 μm pixel−1. The shadow images were then imported into the NRecon program (version 1.7.1.0) to reconstruct cross sections of the specimen. Volume rendering of the reconstructed dataset was completed with CTVox, resulting in a three-dimensional model. Relatively high electron density regions were assigned lower opacity to enhance contrast between the various phases in the sample.

Piercing and Fracture Force Characterization. To assess the piercing force, single MNs loaded with CPO were compressed into a piece of fresh porcine skin using a universal testing machine (ADMET, Norwood, MA) at a jog rate of 1 mm min−1. For the fracture force tests, a single MN was compressed against the flat PMMA surface at a rate of 1 mm min−1. For all experiments, three trials were performed, and the average and standard deviation were reported.

Oxygen Generation Capabilities. To ensure that the CPO within the MNs will produce oxygen when applied, we quantified the oxygen generation capabilities of the MNs in a wound bed model according to the following process. A wound bed phantom made of 2 mm thick 1.5 wt % agarose gel was prepared and placed inside an acrylic chamber. Next, 1 cm circular MN arrays (˜12 MNs) with and without CPO were applied to the agarose gel and sealed to prevent gas exchange between the chamber and the environment. Changes in oxygen levels inside the agarose gel were continuously measured using a RedEye Oxygen sensor patch (Lightwind, USA). The apparatus was closed to the environment for the duration of the experiment (about 6 h).

Planktonic Antibacterial Studies. Antimicrobial properties of CPO-loaded MNs were determined through the time-kill test, inhibition zone measurements, and the LIVE/DEAD assay. Staphylococcus aureus (ATCC 25923) and Pseudomonas aeruginosa (ATCC 25668) were cultured overnight in tryptic soy broth (TSB) (Sigma-Aldrich, St. Louis, MO) and incubated at 37° C. while being agitated. The bacterial count was adjusted to ˜108 CFU/mL, which was used to determine the antimicrobial properties of the samples. For the time-kill test, PET film (control), MNs, and MNs loaded with CPO were housed in 24-well tissue culture plates with 1 mL of phosphate-buffered saline (PBS) (Sigma-Aldrich, St. Louis, MO). Each well was inoculated overnight with 25 μL of bacterial suspension and incubated at 37° C. 20 μL was withdrawn and transferred to TSB agar plates after serial dilution at regular intervals up to 36 h. The plates were then incubated at 37° C. for 16 h after which the colonies were counted.30 254-35 All experiments were carried out in triplicate to determine the average CFU mL−1.

For the inhibition zone tests, 100 μL of bacterial suspension was spread evenly on TSB agar plates. After 5 min, 8 mm disks of PET film (control), MNs, and MNs loaded with CPO were placed on the plates and incubated at 37° C. for 16 h

Further bacterial viability was qualitatively determined using the LIVE/DEAD BacLight bacterial viability kit (Thermo Fisher Scientific, Waltham, MA). 100 μL of S. aureus exposed to either the PET film (control), MNs, or MNs loaded with CPO was withdrawn at 0 and 24 h, and stained with molecular probes SYTO 9, propidium iodide based on the manufacturer's protocol. In this assay, cells would stained green and red to represent live and dead bacteria, respectively. The stained bacterial cells were observed with an inverted fluorescence microscope (Olympus, Waltham, MA) equipped with a camera under a 40× objective and a 10× optical lens using NIS-Elements D software.

Biofilm Antibacterial Studies. Antibiofilm activity of MNs loaded with CPO was evaluated by the colony biofilm model. Overnight cultures of S. aureus and P. aeruginosa were diluted to 2×105 CFU mL−1 with TSB. Next, 50 μL of the diluted bacterial suspension was inoculated onto sterile 10 mm nitrocellulose disks placed on the surface of tryptic soy agar (TSA) plates. To allow biofilm development on the nitrocellulose membrane, the plates were incubated at 37° C. for 72 h. The developed biofilm was then nourished by aseptically transferring to new TSA plates every day for 4 days.31,38 After maturation of the biofilms, 12 mm disks of MNs loaded with CPO and control samples including PET films, CPO powder, and 12 mm disks of MNs without CPO were placed onto the biofilm using sterile forceps. The quantity of CPO powder in the control was estimated to be the amount of CPO powder present in the sample of MNs loaded with CPO. For all experiments, biofilms without any treatments were used as the negative control. The plates were further incubated at 37° C. for 24 h immediately after each treatment. At the end of the incubation period, each biofilm disk was transferred to conical tubes containing 5 mL of TSB with 0.05% of TWEEN-20 (Sigma-Aldrich, St. Louis, MO) and vortexed for 5 min to detach bacteria from the nitrocellulose membrane. The bacterial suspension was serially diluted and 4 μL from each dilution was transferred into TSA plates under aseptic conditions. The plates were further incubated at 37° C. for 24 h and viable CFU mL−1 were calculated.

Furthermore, the extent of bacterial membrane damage imposed by MNs with and without CPO was determined with the LIVE/DEAD BacLight kit (Thermo Fisher). Staining was performed based on the manufacturer's protocol. Briefly, 100 μL of bacterial suspension recovered from the nitrocellulose membrane was stained with 0.3 μL of SYTO 9/propidium iodide (50:50) for 15 min at room temperature protected from light. The cells were imaged using an inverted epi-fluorescence microscope (Nikon Eclipse TS2 Olympus, Waltham, MA) equipped with a camera (photometric cool snap dyno) under a 40× objective and 10× optical lens using NIS-Elements D software.

Ex Vivo Experiments. For the creation of ex vivo dermal wounds, porcine skin was harvested 15 min after sacrifice according to the regulations and guidelines of the Purdue Institutional Animal Care and Use Committee. The harvested skin was then shaved with a surgical prep razor, cleaned with alcohol wipes, and transported in Hank's buffered salt solution on ice. Next, 5 cm×5 cm squares were subsequently cut with an approximate thickness of 4-6 mm. Using a scraper, a 2 cm×2 cm scar was created in the skin, to mimic an open wound, with a depth of approximately 1.5 mm. Next, 10 μL of a bacterial culture suspension at an OD600 of about 0.2 was immediately added in each wound.39,40 The skin explants were then treated with PET and MNs with and without CPO, placed on TSB agar plates, and incubated for 24 h at 37° C. with 5% CO2 and saturated humidity.

After 24 h incubation, 4.5 mm cutaneous punch biopsies were collected from the center of the infected region within the ex vivo wound model. The extracted biopsy samples were placed in a 15 ml tube with 1 mL of PBS of 0.1% Tween-20, sonicated (30 s, 20 W), and serially diluted and plated to quantify the remaining viable bacterial count within the wound after each treatment.

Cytocompatibility. Human dermal fibroblasts, BJ cells (between passages 7 and 9) were cultured in Dulbecco's modified Eagle's medium (DMEM)/F12 medium (Invitrogen Inc., Carlsbad, CA) supplemented with 10% fetal bovine serum. Cells were seeded at a density of 5000 cells cm−2 for HMS32 on 18 mm glass coverslips placed in 12-well plates and were cultured at 37° C. in a humidified environment (95%) with 5% carbon dioxide as described before.41 On day 6 of culture, by which the fibroblasts have typically differentiated, they were exposed to MNs with and without CPO and PET film (control) for 1 h, 24 h, and 6 days (144 h). Cytocompatibility of the MNs with and without CPO and PET film (control) was tested by immunostaining for apoptotic cells using antibodies against caspase-3 (cell signaling technologies, Boston, MA, 1:400 dilution). Cells post-treatment were fixed with 4% paraformaldehyde (Sigma-Aldrich, St. Louis, MO), permeabilized with 1% Triton, and then processed for immunostaining as described previously.42 341-44 To visualize changes in the phenotype, cytoskeleton was stained with phalloidin (Thermo Fisher; 1:40 dilution), while the nuclei were stained with DAPI (500 μg mL−1). Cells were then mounted on antifade and sealed. Images were recorded using Q-capture image acquisition software linked to a BX170 inverted fluorescence microscope (Olympus, Waltham, MA), with 20× objective (NA=0.45). A total of three technical replicates per condition were evaluated with an average of 100 nuclei scored per replicate.

### Results and Discussion

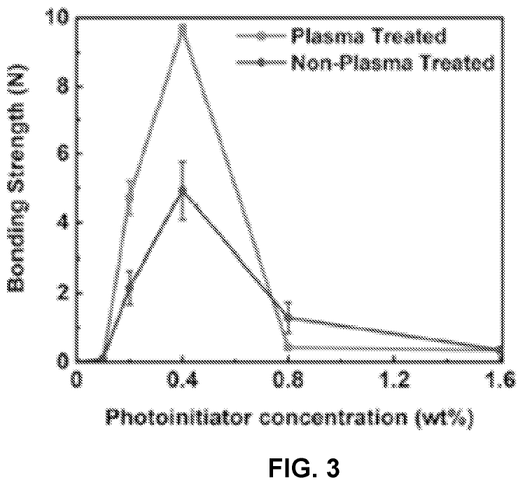

Photopolymerization and Bonding Strength Analysis. By measuring the force to failure for PET bonded to PVP at varying concentrations of photoinitiator, the lowest concentration of photoinitiator with the highest bonding force can be selected. This optimized composition will minimize any toxic effects of the MNs while maintaining strong MN adhesion to the flexible PET substrate. In this experiment, NVP (PVP monomer) with different amounts of photoinitiator ranging between 0 and 1.6 wt % was sandwiched between two PET films that were either plasma treated or not plasma treated. The plasma treatment of the PET substrate was chosen as an additional parameter to investigate, as the generation of additional hydroxyl groups on the surface of the PET should further increase the strength of the chemical bonding between the PET and MNs during the photopolymerization process. As shown in FIG. 3, the plasma-treated samples with 0.4 wt % photoinitiator have the highest bonding force of any sample tested. In all these samples, the PET failed before the interfacial bond.

Although the failure force of 9.66±0.14 N (9.66 MPa) is lower than the expected tensile strength of PET, this is attributed to stress concentrations present at the interface between to two PET film during the T-peel test. Further evidence of this was seen where the applied load fully stretches the PET film at the maximum applied load. The point where the PET fails is the sharp corner at the bonded area, thus indicating the failure of the PET film occurs before any signs of delamination between the two bonded sections on the PET film. In contrast, samples with both higher and lower contents of photoinitiator resulted in a significantly weaker bonding and were easily separated with minimal force. The peel test results show that an optimal amount of 0.4 wt % photoinitiator was needed for creating strong bonds between the photopolymerized PVP with the PET substrate. Samples with 0.2 wt % photoinitiator also developed relatively strong bonds, therefore, if the 0.4 wt % samples prove to be toxic to cell cultures, the 0.2 wt % samples are also another viable option with potential lower toxic effects. Once the photoinitiator concentration is increased to 0.8 and 1.6 wt %, the samples experienced different methods of failure than the 0.4 wt % samples. An experimental sample showed residual PVP on the surface of both pieces of PET. This can be attributed to the difference in mechanical properties of PVP resulting from higher photoinitiator concentrations causing shorter polymer chain lengths and a more brittle mechanical characteristic. Hence, in this application, we will proceed with 0.4 wt % photoinitiator in the fabrication of the MNs

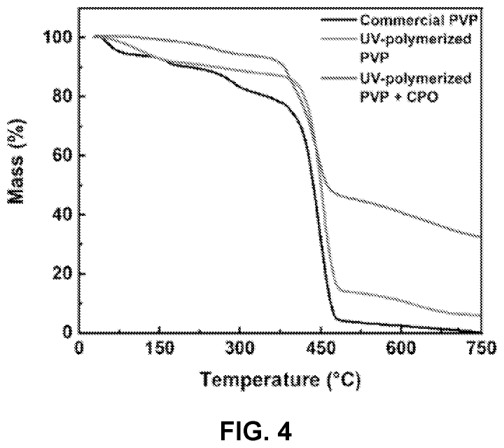

Thermogravimetric Analysis. TGA was performed on commercially purchased PVP and compared to the UV-polymerized PVP with and without CPO loading. Due to the low boiling point of NVP as compared to the polymerized PVP, any significant weight loss in the PVP polymer samples at temperatures below its pyrolysis would indicate the residual monomer inside the PVP polymer. As such, fully polymerized PVP powder with an average molecular weight of 29,000 was used as control in this TGA analysis. As shown in FIG. 4, the commercially purchased PVP exhibits three drops of a few percent that are not present in either of the UV-polymerized samples. The only decrease present in the UV-polymerized that is not present in the commercial sample was at about 100° C., thus indicating that there was likely residual water in the sample. All samples showed a significant weight loss occurring at about 450° C., which is from the pyrolysis of the PVP. Because there is no notable difference in the TGA profile of the UV-polymerized samples and the commercial PVP sample, the established fabrication process confirms the full polymerization of the NVP monomer in the utilized UV polymerization process.

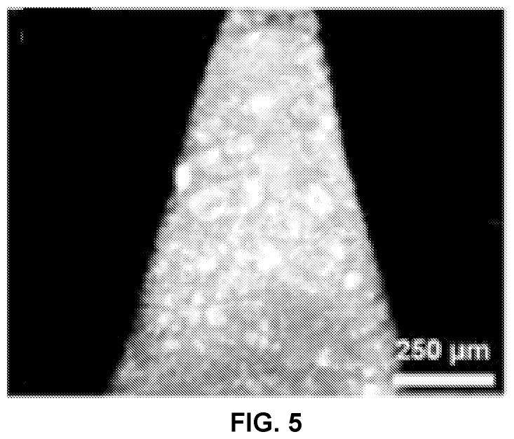

MN Characterization. After confirming complete polymerization of NVP, we investigated the morphology of the MNs and the distribution of CPO particles throughout the MNs. In FIG. 5, the distribution of CPO in a single MN is shown through micro-CT imaging, where the CPO particles are shown in white and light gray, while PVP is relatively transparent. Because the CPO is densely packed in the MN, the PVP mostly serves as a binder between the CPO particles.

At the current concentration of CPO, the entire MN was filled with CPO. However, if less CPO is desired, one simply needs to lower the loading concentration of CPO during the MN fabrication process. The PET substrate enables high flexibility in the MN patch without individual MNs delaminating from the surface. This flexibility allows the patch to follow the contours of the body, thus enabling the use of the patch over the entire body. It is important that the MNs are not too large that they disturb nerve endings and that they are sharp enough to pierce skin with minimal force causing discomfort to the user. The microscopy image of the MNs was analyzed using ImageJ to determine their feature sizes, which resulted in 42.79±2.69 μm, 1.79±0.03 mm, and 1.20±0.05 mm for curvature, height, and width of the MNs, respectively. From these dimensions, it was determined that the fabrication of MN patches through laser processing and soft lithography allows for production of uniform, sharp MNs with potential use in wound treatment applications. Additionally, it allows for easy adjustments to the MN dimensions. For example, the MN patch may be designed with a broad base to increase the surface area for stronger bonding to the PET substrate.

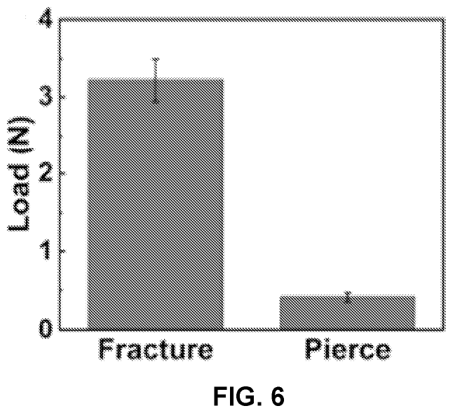

Mechanical Properties of MNs. After examining the physical properties of the MN patch, it was necessary to measure the mechanical properties of the MNs, namely their force to fracture and piercing force. To assess the piercing force, a single MN was compressed into a piece of porcine skin at a rate of 1 mm min−1, as shown in FIG. 6. A piercing force of 0.42±0.05 N was observed, which is comparable to many other MN structures with a similar radius of curvature value between 0.3 and 3 N per MN.45,46 To better visualize the piercing behavior of a full MN patch, an array of 30 MNs was loaded with methylene blue dye and applied to a piece of porcine skin. All 30 MNs successfully pierced the skin and dissolved even with limited moisture present in the skin. Cross-sectional images were taken of the porcine skin to illustrate the diffusion of the dye after the MNs dissolve. The dye diffused into the tissue from the insertion site within 20 min of applying the MN patch, suggesting that therapeutics of a similar molecular size will diffuse in a similar manner into the tissue. It was important that the MNs do not deform or fracture before piercing into skin. Therefore, the fracture force (FIG. 6) was assessed by compressing a single MN into PMMA at a rate of 1 mm min−1. The point at which the MN fractures was taken to be when the force noticeably drops, resulting in an average fracture force of 3.22±0.28 N and thus a factor of safety of 7.75. These results confirm that there is very low risk of MNs fracturing prior to puncturing the skin.

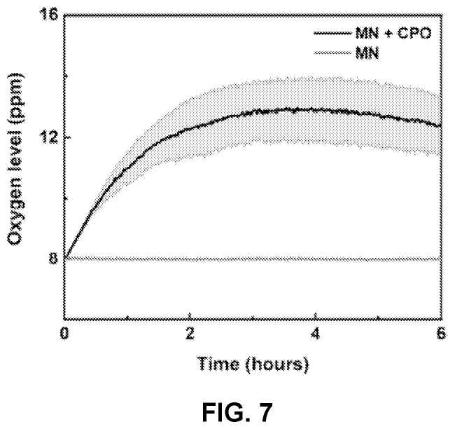

Oxygen Generation Capabilities. To assess how long the CPO particles remain active after insertion into the tissue, the oxygen release characteristics were measured as a function of time in a tissue phantom. FIG. 7 shows the oxygen generation characteristics of a MN array loaded with CPO. In this experiment, 1 cm circles (˜12 MNs) were cut from the larger patch and applied to 1.5 wt % agarose gel. The results, shown in FIG. 7, indicate no change in oxygen levels in the tissue phantom by applying MNs alone without the CPO loading (negative control). However, a steady increase in the oxygen level of up to 12.01±1.02 ppm within 2 h was observed with MNs loaded with CPO. Although PVP MNs rapidly dissolved, the oxygen levels remained elevated for over 6 h in the tissue phantom. This can be attributed to the slow reaction time of CPO and the time it takes for the generated oxygen to diffuse into the surrounding environment. In general, when CPO comes in contact with water, it decomposes into hydrogen peroxide as an intermediate compound and then into oxygen. Due to this two-step degradation process, even though the PVP matrix of the MN dissolves rapidly, there is no burst release of oxygen; instead, there is a sustained supply over a 6 h period. This gradual release not only provides a continuous antibacterial property, but also prevents toxicity caused by high concentrations of oxygen and hydrogen peroxide. 3.6. Planktonic Bactericidal Characterization. Because the time frame for degradation of CPO in the MN patch has been established, it is imperative to quantify the antibacterial efficacy of the MN patch loaded with CPO when exposed to body-like conditions in both planktonic bacteria and biofilms.

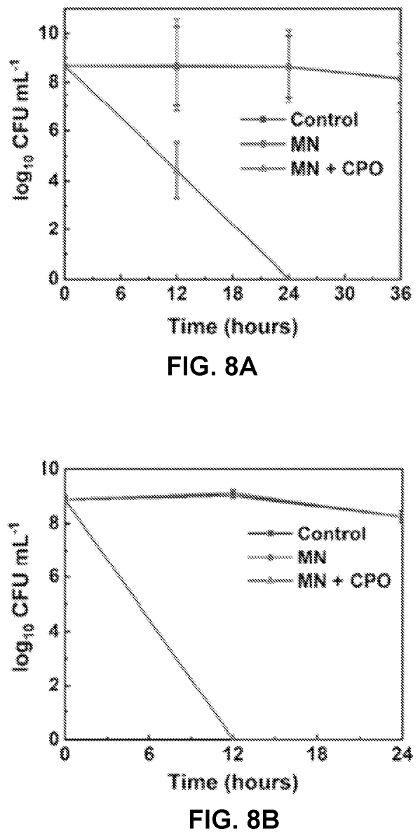

For this test, S. aureus and P. aeruginosa were chosen as the representative Gram-positive and Gram-negative bacterial strains because they are predominant biofilm formers observed in chronic wounds. 512 47 The antibacterial properties of MN patches on planktonic bacteria were assessed through the time-kill test, agar disk diffusion test, and LIVE/DEAD assay (FIG. 8A-B). The antibacterial properties of the MNs in a liquid culture were quantified through a time-kill test and a LIVE/DEAD assay. In this experiment, the MNs with CPO eradicated all the bacteria in the solution within 24 h while there was no significant reduction in bacterial count in the samples without CPO, as shown in FIG. 8A. Similarly, as shown in FIG. 8b, the MNs loaded with CPO rapidly killed P. aeruginosa in a liquid culture. All bacteria in the culture were killed within 12 h, a faster rate than that of S. aureus. The PET films and MNs without CPO both did not significantly influence the bacterial count of P. aeruginosa. The MNs loaded with CPO required more time to induce complete clearance of S. aureus burden, which could be due to the bacterial production of catalase that would in turn, limit the efficacy of hydrogen peroxide against the bacterial cells. To 530 qualitatively determine the extent of membrane damage caused by the MN patch, a representative LIVE/DEAD assay was performed on PET films, MNs without CPO, and MNs with CPO against S. aureus. The bacterial cells were observed to remain green before and after 36 h exposure to PET films and MNs without CPO, thus indicating no signs of membrane damage. However, all the cells were red indicating dead or membrane damaged to S. aureus after 36 h of exposure to MNs with CPO. These results confirm that CPO within the MN maintain their antibacterial properties in liquid culture through rupturing the membrane of bacteria. Although the majority of bacterial communities in biofilms are not planktonic, there would still be planktonic bacteria beneath the biofilm that would be necessary to remove.

It is important for the antibacterial properties to be diffusive across a solid as it ensures the MNs will not only effect local bacteria in a biofilm, but also be broadly bactericidal. To assess the diffusive bactericidal capabilities, agar disk diffusion tests were performed upon PET films, MNs without CPO, and MNs with CPO. No inhibition zones were observed with PET (control) and MN without CPO. The average inhibition zone for 8 mm disks of MNs with CPO (˜4 MN) was 17.00±1.93 mm against S. aureus and 14.60±0.46 mm against P. aeruginosa. Therefore, only the CPO-loaded MNs show a diffusive contact bactericidal property in both Gram-positive and Gram-negative.

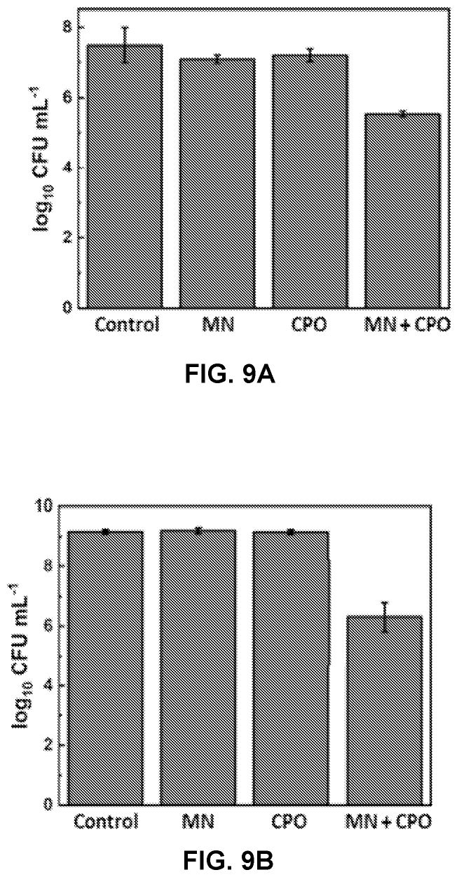

Antibiofilm Activities. Due to the enhanced antibiotic resistance of bacteria in biofilms, it is necessary to also assess the antibacterial properties of the MN patch loaded with CPO against biofilms. Notably, CPO by itself did not affect the number of colonies (FIG. 9a). Therefore, the inherent antibacterial resistance resulting from the architecture of the biofilm was observed to impede the efficacy of the antibacterial properties of the CPO. However, in contrast to CPO powder, MNs loaded with CPO resulted in a significant reduction of the bacterial count in the mature biofilm formed by S. aureus and P. aeruginosa. MNs loaded with CPO generated 2 log 10 reduction in S. aureus mature biofilm cells relative to the control, while they resulted in 2.86 log₁₀ reduction of P. aeruginosa biofilm cells, FIGS. 9A-B. Although MNs with CPO did not completely kill all the bacteria in the biofilm, it significantly reduced the bacterial burdens after one single application. In the clinical settings, the patch would be applied multiple times, which could lead to further complete eradication of biofilm cells.

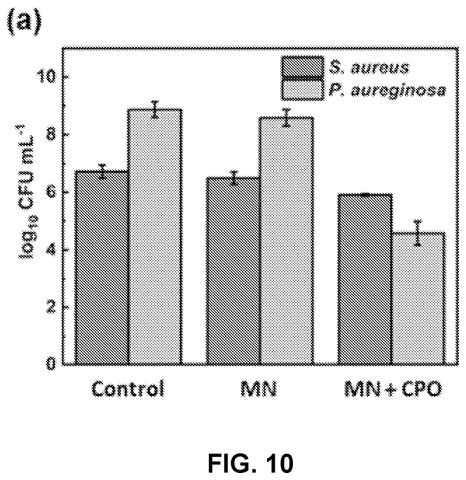

Ex Vivo Assay. To better understand the behavior of the developed MN patch in the intended application, we performed ex vivo antibacterial assays using both P. aeruginosa and S. aureus. As shown in FIG. 10, the MNs with CPO caused more than 4 log₁₀ reduction in CFU for P. aeruginosa and 0.81 log 10 reduction for S. aureus. The smaller decrease in bacterial viability in the S. aureus sample can be attributed to the production of catalase, which limits the influence of CPO. However, in the potential clinical practice, the patch can simply be applied multiple times to completely eradicate the bacteria. It was observed that there is almost no change between the porcine samples for both PET and MNs without CPO. However, when the MN with the CPO sample is applied, there is visible evidence of the degradation of MNs and CPO release into the wound site

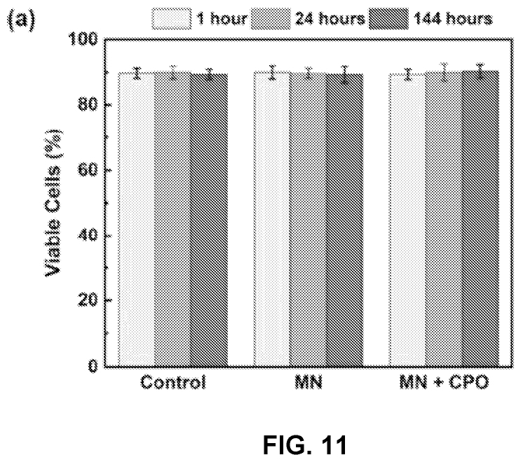

Cytocompatibility. Although the developed MN patch has been shown to be effective against mature biofilms, it needs to be cytocompatible, as solid peroxides, such as CPO, while useful in tissue engineering, 51 can be toxic in high concentrations. To verify this, a caspase-3 assay was performed using dermal fibroblast BJ cells with PET films, MNs without CPO, and MNs loaded with CPO. The average viable cell count from the caspase-3 assay shows an average of ˜90% cell survival across all samples tested at the different time points of 1, 24, and 144 h (6 days) postexposure (FIG. 11). The 1 h time point was chosen because dermal cells quickly react to changes in the environment if there was any injury or damage to the cells due to acute exposure, the fibroblasts would recover in 24 h. By monitoring for an additional 6 days, it shows that there is no long-term exposure effect on dermal cells due to the materials in the samples. Interestingly, as shown in FIG. 11, the presence of CPO did not influence cell viability at any of the chosen time points, which suggests that there is no risk of cell damage due to application of the MNs loaded with CPO. Additionally, the cytocompatibility was assessed qualitatively through immunofluorescence images. Comparing PET and MNs without any CPO, dermal cells did not show any significant difference in the cell phenotype when exposed to MNs loaded with CPO. Hence, the MN patch with CPO shows strong cytocompatibility.

### Observations

Current treatment methods for chronic wounds involve the removal of biofilms requiring repeated treatments that are painful and expensive. Here, we have developed a low cost, flexible, biodegradable, and scalable MN patch loaded with CPO to remove biofilms without inducing pain to the user or generating hazardous waste. The bacterial studies show that MNs loaded with CPO eradicated all planktonic bacteria in a 634

liquid culture within 24 h and possessed strong diffusive bactericidal qualities. When applied to a mature biofilm, there was about a 2 log 10-reduction and a 2.5 log₁₀-reduction in the bacterial count, relative to the control, after a single application of the MN patch loaded with CPO when exposed against S. aureus and P. aeruginosa, respectively. In addition to the bactericidal qualities against biofilms, the developed MN patch provides oxygen to directly combat wound hypoxia. The results from the ex vivo assay indicate that the developed patch possesses the ability to effectively remove bacterial colonies from a wound environment similar to the real application. Moreover, over 90% of cells were continuously viable after exposure to MNs loaded with CPO for 6 days verifying the cytocompatibility of the patch. Further studies should be carried out to determine the efficacy of the patch in in vivo animal models before its wide clinal practices.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

What is claimed is:

1. A medical patch comprising:
a flexible substrate; and
a plurality of water-soluble microneedles extending from a surface of the substrate, the water-soluble microneedles comprising a substance for wound treatment.

2. The medical patch of claim 1, wherein the substance for wound treatment comprises calcium peroxide.

3. The medical patch of claim 1, wherein the water-soluble microneedles comprise:
polyvinylpyrrolidone (PVP).

4. The medical patch of claim 3, wherein the PVP is UV cured from N-vinylpyrrolidone (NVP).

5. The medical patch of claim 1, wherein the substrate is flexible.

6. The medical patch of claim 1, wherein the substrate comprises polyethylene terephthalate (PET).

7. The medical patch of claim 1, wherein the substance is antibacterial.

8. The medical patch of claim 1, wherein application of the medical patch to a wound causes the microneedles to dissolve from water in the wound and the substance to be released and eliminate bacteria.

9. The medical patch of claim 1, wherein the microneedles have a height of 0.5-2.5 mm.

10. A method, comprising:
applying mixture of calcium peroxide (CPO), N-vinylpyrrolidone (NVP), and a photo initiator to a micro-needle mold;
applying a substrate on top of the mixture; and
curing the mixture with ultraviolet light to form an array of microneedles defined by the mold, each of the microneedles comprising CPO and PVP cured from the NVP.

11. The method of claim 10, wherein the mold comprises polydimethylsiloxane (PDMS).

12. The method of claim 10, wherein the mold comprises holes that defined the microneedles, the holes having a depth between 0.5 mm and 2.5 mm.

13. The method of claim 8, further comprising:
applying a silane to the micro-needle mold before applying the mixture.

14. The method of claim 13, further comprising:
reapplying silane to the mold to reuse the mode for forming another microneedle array.

15. The method of claim 10, wherein the microneedles have a height of 0.5-2.5 mm.

16. A medical patch, comprising:
a flexible substrate; and
a plurality of water-soluble microneedles extending from a surface of the substrate, the water-soluble microneedles comprising polyvinylpyrrolidone (PVP) and calcium peroxide (CPO),
wherein application of the medical patch to a wound causes the PVP to dissolve and the CPO to interact with the water and form hydrogen peroxide and oxygen.

17. The medical patch of claim 16, wherein the substrate is flexible.

18. The medical patch of claim 16, wherein the substrate comprises polyethylene terephthalate (PET).

19. The medical patch of claim 16, wherein the PVP is UV cured from N-vinylpyrrolidone (NVP).

20. The medical patch of claim 16, wherein the microneedles have a height of 0.5-2.5 mm.
